(19) United States
(12) Patent Application Publication
(10) Pub. No.: US 2026/0363796 A1
(43) Pub. Date: Nov. 19, 2026

(54) FOUR-DEGREE-OF-FREEDOM MICRO SURGICAL ROBOT WITH REMOTE CENTER OF MOTION AND CONTROL SYSTEM

(71) Applicant: ZHEJIANG UNIVERSITY, Hangzhou (CN)

(72) Inventors: Bo FENG, Hangzhou (CN); Yide LIU, Hangzhou (CN); Shaoxing QU, Hangzhou (CN)

(73) Assignee: ZHEJIANG UNIVERSITY, Hangzhou (CN)

(21) Appl. No.: 19/698,957

(22) Filed: Jun. 5, 2026

Related U.S. Application Data
(63) Continuation of application No. PCT/CN2023/136656, filed on Dec. 6, 2023.

(30) Foreign Application Priority Data
Dec. 5, 2023 (CN) 202311655583.7

(51) Int. Cl. A61B 34/30

(52) U.S. Cl. CPC A61B 34/30; A61B 2034/302

(57) ABSTRACT

A four-degrees-of-freedom (4DoF) miniature surgical robot with a remote center-of-motion (RCM) includes a moving platform mechanism fabricated from a layered composite material by planar processing and at least two limb mechanisms, where the moving platform mechanism includes a central link having a polygonal planar shape and limb links connected to sides of the central link via hinges; the central link is configured as a moving platform for mounting a surgical instrument; a number of the limb links and a number of sides of the polygon are not less than a number of the limb mechanisms; and the limb mechanisms share a same configuration and are each formed by mating assembly of an upper limb and a lower limb. The 4DoF miniature surgical robot satisfies the requirements for high degrees of freedom, high-precision operation, and large working range, and reduces the volume, weight, and manufacturing cost of miniature operating equipment.

### CROSS REFERENCE TO THE RELATED APPLICATIONS

This application is the continuation application of International Application No. PCT/CN 2023/136656, filed on Dec. 6, 2023, which is based upon and claims priority to Chinese Patent Application No. 202311655583.7, filed on Dec. 5, 2023, the entire contents of which are incorporated herein by reference.

### TECHNICAL FIELD

The present disclosure belongs to the field of miniature surgical robots, and specifically relates to a four-degrees-of-freedom (4DoF) miniature surgical robot with a remote center-of-motion (RCM).

### BACKGROUND

Surgical robots can enhance the operational precision and suppress surgeons' hand tremors, thereby improving the outcomes of minimally invasive surgery. Minimally invasive surgery features limited working space, stringent precision requirements, high maneuverability, and high precision in force and displacement control. Taking ophthalmic surgery as an example, it demands precision on the order of 10 micrometers. Any accidental damage to ocular tissues will cause irreversible consequences, which imposes extremely high demands on the stability and precision of surgeons' hands. Accordingly, ophthalmic procedures rank among the most challenging operations in minimally invasive surgery. The human hand has inherent limitations in dexterity, tremor control, and positioning precision. Furthermore, conventional manual minimally invasive surgery is associated with drawbacks such as lengthy training cycles and a high incidence of postoperative complications. Therefore, the development of surgical robots to assist and replace surgeons in conducting contact-based minimally invasive procedures boasts extremely broad application prospects and significant clinical value.

Remote center-of-motion (RCM) is an important concept in robotic surgery. It refers to a fixed point in space around which a surgical instrument can pivot. This point is usually located at or near an entry port where the surgical instrument enters the patient's body, so RCM is well suited for performing minimally invasive surgery. The concept of RCM can be used to design robotic surgical systems that deliver high precision, stability, and safety.

Miniature surgical robots are a new type of robotic technology, generally referring to robots with a dimension of less than 5 cm and configured to perform surgical tasks. These robots feature small volume, lightweight, high maneuverability, high operational precision, and easy integration. Compared with large surgical robots, miniature surgical robots have higher precision and better responsiveness. Taking ophthalmic surgery as an example, miniature surgical robots can overcome many difficulties of intraocular contact-based operations, greatly assist surgeons in surgical procedures, reduce the difficulty of surgical training, improve treatment outcomes, and reduce postoperative complications. However, due to dimensional constraints, conventional methods cannot achieve the design and fabrication of miniature robots. Currently, planar design and fabrication methods are generally adopted for miniature robots.

Planar processing refers to processing methods for planar materials, including but not limited to additive fabrication methods such as three-dimensional (3D) printing, printing, spraying, extrusion, and chemical synthesis, or subtractive fabrication methods such as cutting, wire cutting, laser ablation, photolithography, and chemical etching. Planar materials refer to materials with their thickness much smaller (at least one order of magnitude) than their length and width, including but not limited to rigid planar materials such as metal sheets, plastic sheets, wood sheets, and carbon fiber sheets, and flexible planar materials such as polymer films, gel layers, textiles, and metal foils.

In view of the miniature surgical robots, due to limitations in the design methods and fabrication processes, conventional surgical robots are difficult to reduce to a centimeter-scale volume while also achieving micrometer-level operational precision. Existing surgical robot systems, although capable of achieving micrometer-level operational precision, still suffer from large volume, difficult deployment, and high cost relative to their working range. Moreover, there is currently no miniature surgical robot system that can realize the RCM function while possessing in-situ puncture capability.

### SUMMARY

An objective of the present disclosure is to overcome the deficiencies of the prior art regarding the operational precision and working range of miniature surgical robots, and to provide a 4DoF miniature surgical robot with a remote center-of-motion (RCM). The 4DoF miniature surgical robot satisfies the requirements for high degrees of freedom, high-precision operation, and large working range, reduces the volume, weight, and manufacturing cost of miniature operating equipment, and realizes 4DoF operation with integrated rotation and in-situ translation. Meanwhile, the miniature surgical robot achieves operational precision on the order of 10 micrometers and a working space on the order of 10 cubic centimeters.

It is to be noted that, in the present disclosure, the concept of degrees of freedom refers to the motions executable by the robot's moving platform and a surgical actuator mounted thereon in space. In the 3D space defined by the Cartesian coordinate system (OXYZ), a rigid body has a maximum of six degrees of freedom, namely translation along the X, Y, and Z axes and rotation about the X, Y, and Z axes. The four degrees of freedom of the moving platform of the miniature surgical robot proposed in the present disclosure refer to the capabilities of rotating about the X, Y, and Z axes and translating along the Z axis.

Specifically, the present disclosure adopts the following technical solutions.

In a first aspect, the present disclosure provides a 4DoF miniature surgical robot with an RCM, including a moving platform mechanism fabricated from a layered composite material by planar processing and at least two limb mechanisms, where
the moving platform mechanism includes a central link having a polygonal planar shape and limb links connected to sides of the central link via hinges; the central link is configured as a moving platform for mounting a surgical instrument; a number of the limb links and a number of sides of the polygon are not less than a number of the limb mechanisms;
the limb mechanisms share a same configuration and are each formed by mating assembly of an upper limb and a lower limb;
the lower limb is integrally formed by a sixth link and a seventh link; the sixth link includes a main body portion and a protruding portion; the main body portion is continuously formed by a first straight section, an annular section, and a second straight section, all having the same width; the seventh link is connected to an outer end portion of the first straight section fixedly, or rotatably via a fifth hinge; the protruding portion is provided on an outer annular side of the main body portion and is provided with a mounting hole connectable to an external drive mechanism; and a straight line defined by a side of the second straight section away from the annular section is configured as a rotation center axis of the lower limb when the lower limb is driven;
the upper limb is integrally formed by a tenth link, an eleventh link, a twelfth link, and a thirteenth link; the tenth link is connected to one end of the eleventh link fixedly, or rotatably via a sixth hinge; the other end of the eleventh link is rotatably connected to one end of the twelfth link via a seventh hinge; the other end of the twelfth link is rotatably connected to the thirteenth link via an eighth hinge; and the thirteenth link is fixedly assembled with a corresponding limb link on the moving platform mechanism via male and female connectors;
the lower limb and the upper limb are fixedly assembled via a set of male and female connectors respectively provided on the seventh link and the tenth link;
the limb mechanisms assembled on the central link satisfy following five constraints:
a first constraint: each limb mechanism includes at least one of the fifth hinge and the sixth hinge; and, for any limb mechanism that includes both hinges, axes of the fifth hinge and the sixth hinge coincide with each other;
a second constraint: axes of the seventh hinge and the eighth hinge of each of the limb mechanisms are perpendicular to each other;
a third constraint: a second attitude angle and a third attitude angle of each of the limb mechanisms are less than 18°;
a fourth constraint: the eighth hinge in each of the limb mechanisms is parallel to a hinge on the moving platform that is connected to a current limb mechanism; and
a fifth constraint: all first center points and all second center points of all the limb mechanisms coincide at one and the same point; and
in each of the limb mechanisms, the provided fifth hinge or sixth hinge is configured as a designated hinge, an intersection point of the designated hinge and the rotation center axis is configured as the first center point, an intersection point of the designated hinge and the seventh hinge is configured as the second center point, an angle between the designated hinge and the rotation center axis is configured as the second attitude angle, and an angle between the designated hinge and the seventh hinge is configured as the third attitude angle.

Preferably, in the first aspect, in the moving platform mechanism, the central link is a fifth link shaped as a rectangle; the fifth link is connected to four limb links: a first link, a second link, a third link, and a fourth link; one end of the first link, one end of the second link, one end of the third link, and one end of the fourth link are respectively rotatably connected to the fifth link via a first hinge, a second hinge, a third hinge, and a fourth hinge, and the other end of the first link, the other end of the second link, the other end of the third link, and the other end of the fourth link are respectively fixedly connected to a first limb mechanism, a second limb mechanism, a third limb mechanism, and a fourth limb mechanism in a manner of assembling male and female connectors; and the first hinge and the third hinge are parallel to each other, the second hinge and the fourth hinge are parallel to each other, and the first hinge and the second hinge are perpendicular to each other; and
in the moving platform mechanism and the four limb mechanisms connected to the moving platform mechanism, a combined mechanism of the first link and the first limb mechanism and a combined mechanism of the third link and the third limb mechanism are symmetrically disposed on two sides of the moving platform, and a combined mechanism of the second link and the second limb mechanism and a combined mechanism of the fourth link and the fourth limb mechanism are symmetrically disposed on two sides of the moving platform.

Preferably, in the first aspect, a robot body assembled on four rotary drive mechanisms satisfies following four constraints:
a first constraint: the lower limb and the upper limb of each of the four limb mechanisms have a same equivalent radius;
a second constraint: the first limb mechanism and the third limb mechanism have a same second attitude angle, third attitude angle, first hinge spacing, and second hinge spacing, and the second limb mechanism and the fourth limb mechanism have a same second attitude angle, third attitude angle, first hinge spacing, and second hinge spacing;
a third constraint: the first hinge spacing and the second hinge spacing of each of the four limb mechanisms are 0.2 to 2 times the equivalent radius; and
a fourth constraint: the second attitude angle and the third attitude angle of each of the four limb mechanisms are less than 90°; and
in each of the limb mechanisms, a distance from the first center point to a farthest end of the designated hinge, or a distance from the first center point to a farthest end of the side of the second straight section away from the annular section, is configured as an equivalent radius of the lower limb; a distance from the second center point to the eighth hinge is configured as an equivalent radius of the upper limb; an angle between the rotation center axis and a horizontal plane is configured as the first attitude angle; a distance from the eighth hinge to the hinge on the moving platform that is connected to the current limb mechanism is configured as the first hinge spacing; and half of a distance from the hinge on the moving platform that is connected to the current limb mechanism to a symmetric hinge is configured as the second hinge spacing.

Preferably, in the first aspect, all links of the 4DoF miniature surgical robot are made of a layered composite material; the layered composite material includes a flexible planar material layer configured as an intermediate layer and rigid planar material layers located on two sides of the intermediate layer; the flexible planar material layer is bonded and fixed to the rigid planar material layers by an adhesive material layer; at a hinge position between every two adjacent links, the flexible planar material layer is continuous while the rigid planar material layers are discontinuous; and edges of the rigid planar material layers on two sides of a discontinuity position are provided with rectangular grooves at intervals, such that the rigid planar material layers interlock with each other via the sawtooth edges formed by the rectangular grooves and are allowed to freely rotate about a hinge under a connecting action of the flexible planar material layer.

Preferably, in the first aspect, the flexible planar material layer is a flexible polymer film, a flexible gel layer, a flexible textile, or a flexible metal foil; and each of the rigid planar material layers is a rigid metal sheet, a rigid plastic sheet, a rigid glass sheet, a rigid resin sheet, a rigid wood sheet, or a rigid composite material sheet.

Preferably, in the first aspect, in each of the limb mechanisms, male and female connectors for connecting two links are respectively provided on the two links; a male connector of one link is assembled into a female connector of the other link; and the two links are bonded together by adhesive.

In a second aspect, the present disclosure provides a control system for a 4DoF miniature surgical robot with an RCM, including any one of the 4DoF miniature surgical robots in the first aspect and at least four independent rotary drive mechanisms, where
the lower limb of each of the limb mechanisms is provided with one rotary drive mechanism that is fixed to a base for driving the limb mechanism to rotate about the corresponding rotation center axis; and if the number of rotary drive mechanisms is greater than the number of limb mechanisms, the remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle; and the first attitude angle of each of the limb mechanisms is less than 90°.

Preferably, in the second aspect, there are four limb mechanisms and four rotary drive mechanisms, and the rotary drive mechanisms correspond one-to-one with the limb mechanisms to form a driving relationship; and preferably, the four limb mechanisms have the same first attitude angle.

Preferably, in the second aspect, each of the rotary drive mechanisms includes a drive motor and a transmission plate; the transmission plate is assembled on a drive shaft of the drive motor and includes a plate surface perpendicular to the drive shaft; the transmission plate provides a mounting plane for the protruding portion of the sixth link via a boss structure; and preferably, the drive motor is a micro servo motor.

Preferably, in the second aspect, a center of the fifth link configured as the moving platform is provided with a mounting hole, and a surgical instrument is mounted via the mounting hole; and preferably, the surgical instrument includes one or more of a blade, a miniature camera, an injection needle, a lens, and a suction cup.

Compared with the prior art, the present disclosure has the following beneficial effects:
1) The present disclosure provides a 4DoF miniature surgical robot with an RCM. It has a volume of less than 80 cubic centimeters, and is capable of achieving a repeat positioning precision of 10 micrometers and a workspace of 10 cubic centimeters. It provides an RCM function, has a 4DoF motion capability (including three rotational degrees of freedom and one translational degree of freedom), and can realize in-situ puncture.
2) The 4DoF miniature surgical robot of the present disclosure is fabricated from smart composite materials via planar processing, and features a simple fabrication process and low fabrication cost.
3) The robot of the present disclosure has high adaptability for surgical operations. Its replaceable end effector allows the robot to perform tasks such as cutting and puncturing according to different surgical requirements, thereby delivering excellent practical performance.

### BRIEF DESCRIPTION OF THE DRAWINGS

FIG. 1 is a schematic diagram of a theoretical model of a 4DoF miniature surgical robot;

FIGS. 2A-2B are plan views of a moving platform;

FIGS. 3A-3B are plan views of lower limbs of a limb A and a limb C;

FIGS. 4A-4B are plan views of lower limbs of the limb B and a limb D;

FIGS. 5A-5B are plan views of upper limbs of the limb A and the limb C;

FIGS. 6A-6B are plan views of upper limbs of the limb B and the limb D;

FIG. 7 is a schematic diagram showing rectangular grooves in the moving platform as an example;

FIGS. 8A-8B are plan views of the moving platform with an island chain and an upper limb;

FIGS. 9A-9B are plan views of a lower limb with an island chain;

FIG. 10 is a plan view of nine parts required to fabricate the 4DoF miniature surgical robot;

FIGS. 11A-11C are fabrication drawings of the 4DoF miniature surgical robot;

FIGS. 12A-12E are flowcharts of actual fabrication and assembly of the 4DoF miniature surgical robot;

FIGS. 13A-13D are schematic diagrams of the miniature surgical robot and its drive and transmission system;

FIGS. 14A-14D are schematic diagrams of motion degrees of freedom of the moving platform of the miniature robot;

FIGS. 15A-15C are schematic diagrams of the moving platform of the miniature robot provided with three types of instruments: a blade, a miniature camera, and an injection needle;

FIGS. 16A-16B are schematic diagrams of the miniature surgical robot with two limb mechanisms and four motors, and its drive and transmission system;

FIGS. 17A-17B are schematic diagrams of the miniature surgical robot with three limb mechanisms and four motors, and its drive and transmission system;

FIGS. 18A-18B are schematic diagrams of the miniature surgical robot with six limb mechanisms and six motors, and its drive and transmission system; and

FIGS. 19A-19H show comparison of the presence and absence of a fifth hinge and a sixth hinge on a limb mechanism.

### DETAILED DESCRIPTION OF THE EMBODIMENTS

To make the foregoing objectives, features, and advantages of the present disclosure clearer and more comprehensible, the specific implementations of the present disclosure are described in detail below with reference to the drawings. The following describes many details in order to provide a thorough understanding of the present disclosure. However, the present disclosure can be implemented in many other ways other than those described herein, and those skilled in the art can make similar improvements without departing from the connotation of the present disclosure, and thus the present disclosure is not limited to the specific embodiments disclosed below. All the technical features in the embodiments of the present disclosure can be correspondingly combined on the premise of no conflict.

In the description of the present disclosure, it should be understood that the terms “first” and “second” are used only for the purpose of distinguishing description, and shall not be construed as indicating or implying relative importance or implicitly indicating the quantity of the technical features indicated. Therefore, features defined by “first” and “second” may explicitly or implicitly include at least one of the features.

The present disclosure provides a 4DoF miniature surgical robot with a remote center-of-motion (RCM). The 4DoF miniature surgical robot satisfies the requirements for high degrees of freedom, high-precision operation, and large working range, reduces the volume, weight, and manufacturing cost of miniature operating equipment, and realizes 4DoF operation with integrated rotation and in-situ translation. Meanwhile, the miniature surgical robot achieves operational precision on the order of 10 micrometers and a working space on the order of 10 cubic centimeters. In the present disclosure, the concept of degrees of freedom refers to the motions executable by the robot's moving platform and a surgical actuator mounted thereon in space. In the 3D space defined by the Cartesian coordinate system (OXYZ), a rigid body has a maximum of six degrees of freedom, namely translation along the X, Y, and Z axes and rotation about the X, Y, and Z axes. The four degrees of freedom of the moving platform of the miniature surgical robot proposed in the present disclosure refer to the capabilities of rotating about the X, Y, and Z axes and translating along the Z axis.

The 4DoF miniature surgical robot with an RCM provided in the present disclosure includes a moving platform mechanism fabricated from a layered composite material by planar processing and at least two limb mechanisms. Since the robot configurations differ for different numbers of limb mechanisms, but the actuation principle of the robot is substantially the same, the most commonly used form with four limb mechanisms is introduced below.

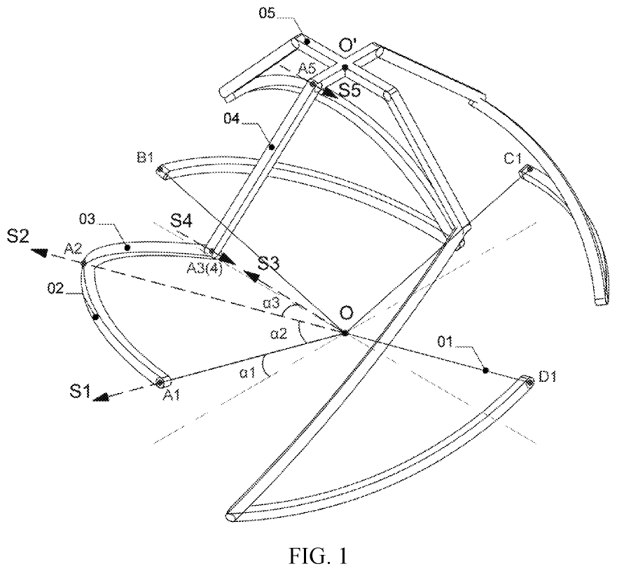

In a preferred embodiment of the present disclosure, a theoretical model of a 4DoF miniature surgical robot with four limb mechanisms is shown in FIG. 1. The mechanism includes one fixed platform, one moving platform, and four limb mechanisms, each limb mechanism including three links. O represents a center of the fixed platform, O′ represents a center of the moving platform, and A, B, C, and D respectively represent the four limb mechanisms. The geometric relationship of the mechanism is illustrated by labeling the limb mechanism A, and the geometric relationships of the other limbs are the same as that of the limb mechanism A. In the figure, the fixed platform is labeled 01, the moving platform is labeled 05, and the three links of the limb mechanism A from the fixed platform to the moving platform are labeled 02, 03, and 04 in sequence. The part 01 and the part 02 are connected by a revolute joint, the part 02 and the part 03 are connected by a revolute joint, the part 03 and the part 04 are connected by a universal joint, and the part 04 and the part 05 are connected by a revolute joint. If the universal joint is considered as two revolute joints, one limb mechanism of the mechanism includes a total of five revolute joints, axes of which from the fixed platform to the moving platform are S1, S2, S3, S4, and S5, respectively. The geometric relationships of the limb mechanism are as follows. Lengths OA1, OA2, OA3, and OA4 are equal to R; an angle between S1 and a horizontal plane is al; an angle between S1 and S2 is a2; an angle between S2 and S3 is a3; S3 is perpendicular to S4, where they may or may not intersect, and in the present disclosure, for fabrication convenience, S3 and S4 intersect at point A3(4); S4 is always parallel to S5; a length of A3(4)A5 is L1; and a distance from A5 to O′ is L2. For each limb mechanism, a straight line can be obtained by projecting the axis S1 of the revolute joint onto a ground, and a direction away from the point O along the straight line is defined as a distribution direction of the limb mechanism. A vector along the direction is defined as a distribution vector of the limb mechanism, and an angle between the distribution vectors of adjacent limb mechanisms is defined as a distribution angle. The distribution angles of the four limb mechanisms of the robot can be arbitrarily selected, provided that the limb mechanisms do not interfere with each other. In the present disclosure, the distribution angles are preferably 90°, and will not be discussed later as a mechanism parameter. For the miniature surgical robot constructed according to the above geometric relationships, a line passing through the point O′ and perpendicular to the moving platform always passes through the point O, and the point O is an RCM point of the mechanism. In actual fabrication, in order to prevent the actuator mounted on the moving platform from being restricted by the fixed platform, the following examples do not use smart composite materials to constrain the positions of the points O, A1, B1, C1, and D1, but use a metal base to constrain the positions of the axes OA1, OB1, OC1, and OD1 in space and ensure that they intersect at the point O, which will be illustrated in the subsequent description.

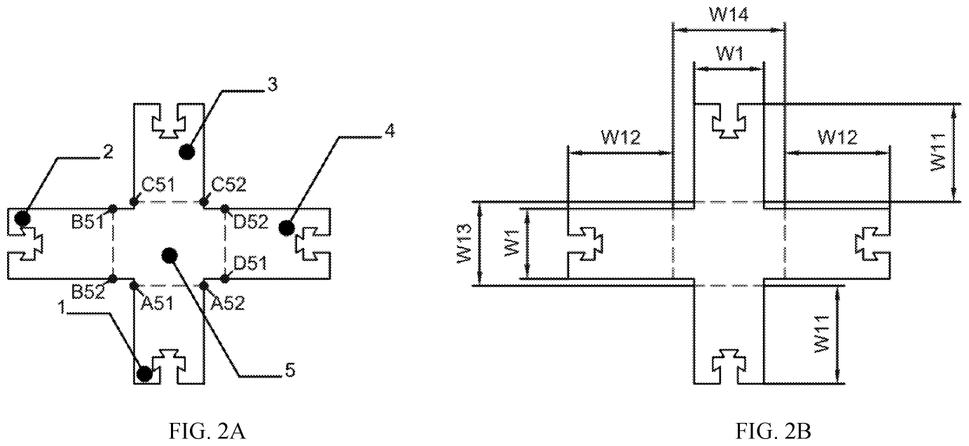

Based on the above theoretical model, in a preferred embodiment of the present disclosure, a smart layered composite material and planar processing technology are used to physically fabricate the robot, thereby designing a 4DoF miniature surgical robot with an RCM. The robot includes one moving platform mechanism fabricated from a layered composite material by planar processing and four limb mechanisms fabricated by planar processing from the layered composite material. The specific implementations are described in detail below with reference to FIGS. 2A to 14D.

In the surgical robot, the limb mechanisms connected to the moving platform mechanism are respectively a first limb mechanism, a second limb mechanism, a third limb mechanism, and a fourth limb mechanism, corresponding to the four limb mechanisms A, B, C, and D in the theoretical model described above. The moving platform mechanism is integrally formed by first link 1, second link 2, third link 3, fourth link 4, and fifth link 5. The fifth link 5 is configured as the moving platform, and a surgical instrument can be mounted at its center. One end of the first link 1, one end of the second link 2, one end of the third link 3, and one end of the fourth link 4 are respectively connected to the fifth link 5 by a first hinge, a second hinge, a third hinge, and a fourth hinge in a rotational manner, and the other end of the first link, the other end of the second link, the other end of the third link, and the other end of the fourth link are respectively fixedly connected to the first limb mechanism, the second limb mechanism, the third limb mechanism, and the fourth limb mechanism in a manner of assembling male and female connectors. In this embodiment, the first hinge and the third hinge are parallel to each other, the second hinge and the fourth hinge are parallel to each other, and the first hinge is perpendicular to the second hinge.

The first limb mechanism, the second limb mechanism, the third limb mechanism, and the fourth limb mechanism have the same configuration, and are each formed by mating assembly of an upper limb and a lower limb. One of the limb mechanisms is configured as an example below to describe the specific configurations of the upper limb and the lower limb.

In an embodiment of the present disclosure, the lower limb is integrally formed by sixth link 6 and seventh link 7. The sixth link 6 includes a main body portion and a protruding portion. The main body portion is continuously formed by a first straight section, an annular section, and a second straight section, all having the same width. The seventh link 7 is connected to an outer end portion of the first straight section by a fifth hinge in a rotational manner. The protruding portion is provided on an outer annular side of the main body portion and is provided with a mounting hole connectable to an external drive mechanism. One side of the protruding portion is collinear with one side of the second straight section, and together they form a rotation center axis of the lower limb when driven.

In an embodiment of the present disclosure, the upper limb is integrally formed by tenth link 10, eleventh link 11, twelfth link 12, and thirteenth link 13. The tenth link 10 and one end of the eleventh link 11 are connected by a sixth hinge in a rotational manner. The other end of the eleventh link 11 is connected to one end of the twelfth link 12 by a seventh hinge in a rotational manner. The other end of the twelfth link 12 is connected to the thirteenth link 13 by an eighth hinge in a rotational manner, and the thirteenth link 13 is connected to the corresponding link of the moving platform mechanism. The axes of the seventh hinge and the eighth hinge are perpendicular to each other.

In an embodiment of the present disclosure, the lower limb and the upper limb are fixedly assembled via a set of male and female connectors respectively provided on the seventh link 7 and the tenth link 10.

In an embodiment of the present disclosure, for ease of mounting and driving, in the 4DoF miniature surgical robot with an RCM, in the moving platform mechanism and the four limb mechanisms connected thereto, a combined mechanism of the first link 1 and the first limb mechanism and a combined mechanism of the third link 3 and the third limb mechanism are symmetrically disposed on two sides of the moving platform, and a combined mechanism of the second link 2 and the second limb mechanism and a combined mechanism of the fourth link 4 and the fourth limb mechanism are symmetrically disposed on two sides of the moving platform. In the 4DoF miniature surgical robot, the four limb mechanisms are driven respectively by four independent rotary drive mechanisms to rotate about their respective rotation center axes of the lower limbs. The specific driving method will be described later and will not be elaborated here. In an embodiment of the present disclosure, all links forming the robot body are made of a layered composite material. The intermediate layer of the layered composite material is a flexible planar material layer, and the two sides of the intermediate layer are rigid planar material layers. The flexible planar material layer is bonded and fixed to the rigid planar material layers by an adhesive material layer. At a hinge position between every two adjacent links, the flexible planar material layer is continuous while the rigid planar material layers are discontinuous. Edges of the rigid planar material layers on two sides of a discontinuity position are provided with rectangular grooves at intervals, such that the rigid planar material layers interlock with each other via the sawtooth edges formed by the rectangular grooves and are allowed to freely rotate about a hinge under the connecting action of the flexible planar material layer.

The layered composite material of the present disclosure can be formed into a multi-layer composite planar structure of smart composite material by planar processing of planar materials combined with an adhesive fabrication process. In this layered composite material, the intermediate layer is a flexible planar material layer, the outermost two sides are rigid planar material layers, and the flexible planar material layer and the rigid planar material layers are fixed by an adhesive material layer. Rigid planar materials include but are not limited to rigid metal sheets, rigid plastic sheets, rigid glass sheets, rigid resin sheets, rigid wood sheets, rigid composite material sheets (e.g., rigid carbon fiber sheets), etc. Flexible planar materials include but are not limited to flexible polymer films, flexible gel layers, flexible textiles, flexible metal foils, etc. The adhesive material layer may be in the form of glue, adhesive, or hot-press tape. After such a layered composite material is subjected to planar processing, the rigid planar material layers in the smart composite material sandwich the flexible planar material layer, and the rigid planar material layers on two sides can be linearly discontinuous at the hinge positions, while the flexible planar material layer in the intermediate remains continuous. Thus, a linear rotation axis, i.e., the hinge, is formed. This linear rotation axis is a rotation mechanism connected by the flexible planar material layer in the form of a straight line, and the rigid planar material layers on two sides, after being separated by planar processing, can rotate about the linear rotation axis using the flexible planar material layer, thereby obtaining motion degrees of freedom and forming the rotational degrees of freedom required for the robot hinges. The rigid planar material layers on two sides of the flexible planar material layer interlock with each other via the sawtooth edges at the hinge position, and are allowed to freely rotate about the hinge under the connecting action of the flexible planar material layer.

Based on the link members formed by such smart composite material, the motion degrees of freedom of the device can be further designed three-dimensionally as required, thereby achieving different actuation functions.

Based on the limb mechanism formed by planar processing, when two links need to be connected, a corresponding set of male and female connectors can be provided at the connection position, and the allocation of the male and female connectors can be adjusted as needed. The male and female connectors for connecting the two links are respectively provided on the two links. The male connector of one link is assembled into the female connector of the other link, and the two links are bonded together by adhesive or the like.

Next, the planar design process and the fabrication and assembly process of the 4DoF miniature surgical robot made of the smart composite material are described. The planar design process requires first determining the geometric characteristics of the robot, and then performing supplementary process design based on the planar design to meet the processing requirements of the smart composite material. The fabrication and assembly process of the smart composite material is to obtain all components constituting the robot by processing and bonding rigid and flexible sheets according to the fabrication drawings generated in the first step, and then assemble the fabricated components to obtain the final 4DoF miniature surgical robot.

In an embodiment of the present disclosure, the planar design process of the 4DoF miniature surgical robot is as follows.

The 4DoF miniature surgical robot is fabricated by assembling one moving platform mechanism and four similarly shaped limb mechanisms. For ease of processing, the moving platform mechanism and each limb mechanism are assembled, and each limb mechanism is also divided into two parts that are assembled.

As described in the theoretical model, to ensure that the mechanism has a large workspace and good transmission performance, limb mechanisms facing each other have the same parameters, and adjacent limb mechanisms have different parameters. The present disclosure uses FIGS. 2A-2B, 3A-3B, 4A-4B, 5A-5B, and 6A-6B to illustrate these parts. In the figures, solid lines represent part outlines, dashed lines represent flexible hinges, the parts are divided into different links by the hinges, and dash-dotted lines are auxiliary lines. FIGS. 2A, 3A, 4A, and 5A label the serial numbers of feature points, and FIGS. 2B, 3B, 4B, and 5B label link dimensions.

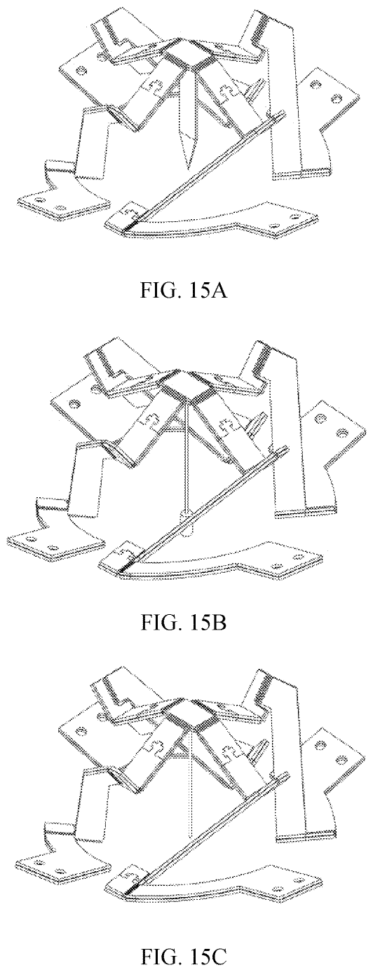

FIGS. 2A-2B are plan views of the moving platform mechanism. The moving platform mechanism includes five links each with a width of W1. The first link 1 and the third link 3 are connected to the limb mechanisms A and C with the same parameters, and their lengths are both W11. The second link 2 and the fourth link 4 are connected to limb mechanisms B and D with the same parameters, and their lengths are both W12. The fifth link 5 is the moving platform, which may be provided with holes to mount different surgical instruments (to be described later, FIGS. 15A-15C). The hinge connecting the fifth link 5 and the first link 1 is A51-A52. The hinge connecting the fifth link 5 and the second link 2 is B51-B52. The hinge connecting the fifth link 5 and the third link 3 is C51-C52. The hinge connecting the fifth link 5 and the fourth link 4 is D51-D52. The distance between the hinge A51-A52 and the hinge C51-C52 is W13, while the distance between the hinge B51-B52 and the hinge D51-D52 is W14. In FIGS. 2A-2B, the first link 1, the second link 2, the third link 3, and the fourth link 4 each include a cross-shaped female connector. The plug-in connectors included in these four links and in the links described below all have the same dimensions, and their shapes ensure the assembly precision of two connected links before adhesive bonding and curing.

As described above, each limb mechanism is divided into two parts that are assembled. An upper half portion of the limb mechanism connected to the moving platform mechanism is called the upper limb, and a lower half portion of the limb mechanism configured as an input end connected to a motor is called the lower limb. For ease of description, the present disclosure stipulates that the labeling of points on the limb mechanism A, the limb mechanism B, the limb mechanism C, and the limb mechanism D starts with their respective representative letters.

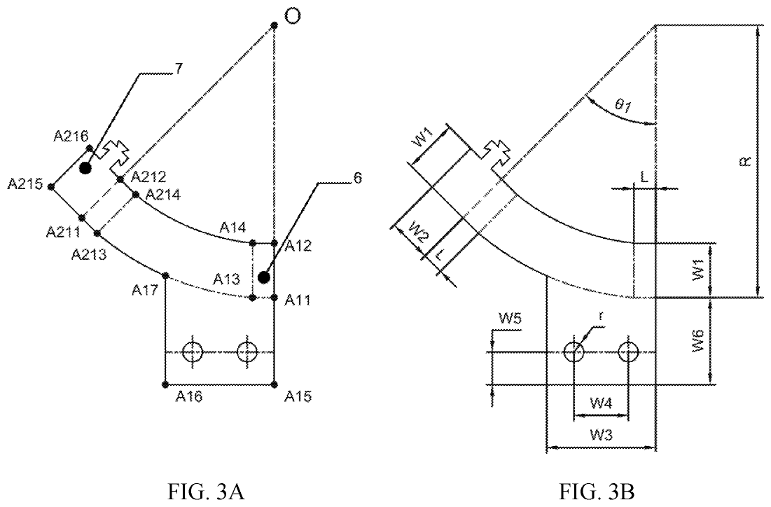

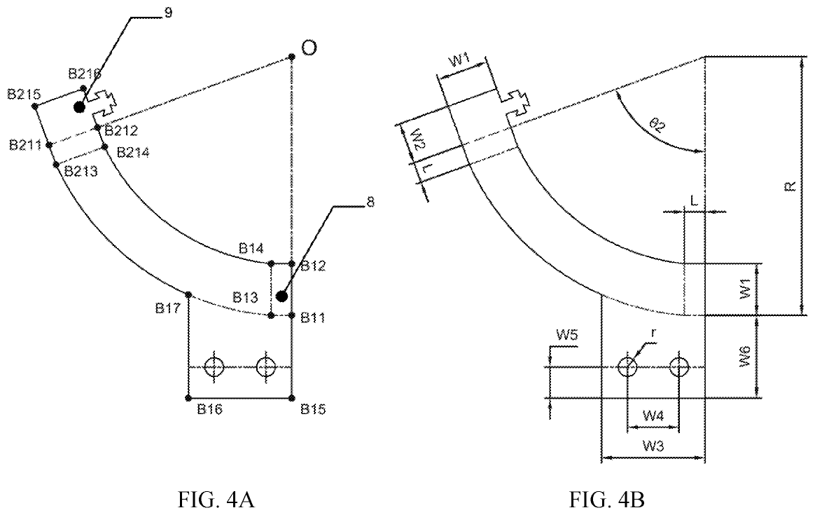

FIGS. 3A-3B are plan views of the lower limb of the limb mechanism A and the limb mechanism C. FIGS. 4A-4B are plan views of the lower limb of the limb mechanism B and the limb mechanism D. FIGS. 3A-3B and 4A-4B have most dimensions the same, with the only difference being the different angles of θ1 and θ2 (θ1 and θ2 correspond to a2 in the theoretical model). Therefore, only FIG. 3A-3B are used to introduce the lower limb.

In FIG. 3A-3B, the lower limb includes the sixth link 6 and the seventh link 7. The sixth link 6 includes a main body portion and a protruding portion, and the main body portion is continuously formed by a first straight section, an annular section, and a second straight section, all having the same width. The design method is as follows. Starting from the center point O, radius R is drawn downward to point A11. An upward dimension equal to the link width W1 is measured from the point A11 to obtain point A12. Solid line A11-A12 is rotated clockwise by angle 01 about O to form hinge A211-A212. The dashed line A211-A212 is translated to the right by distance L along a direction perpendicular to dash-dotted line O-A212, and dash-dotted line A213-A214 is thus obtained. The solid line A11-A12 is translated to the left by distance L along the direction perpendicular to the dash-dotted line O-A12, and dash-dotted line A13-A14 is thus obtained. A solid arc centered at O is drawn to connect point A214 and point A14. A dash-dot arc centered at O is drawn to connect point A213 and point A13. A downward extension of W6 is made from the point A11 to obtain point A15. A leftward extension of W3 is made from the point A15 to obtain point A16. An upward extension from the point A16 intersects the arc A213-A11 at point A17. A solid arc centered at O is drawn to connect the point A213 and the point A17. With reference to line A15-A16, two circular holes, each having radius r, are formed with a horizontal center-to-center spacing W4 and with their centerline located at a vertical distance W5 from line A15-A16, for passage of bolts connecting to the motor. The design method of the seventh link 7 is as follows. The dashed line A211-A212 is translated to the left by distance W2 along the direction perpendicular to the dash-dotted line O-A212, and solid line A215-A216 is thus obtained. Solid lines are drawn to connect point A215 to point A211 and point A215 to point A216. A cross-shaped male connector is disposed between the point A216 and point A212. Thus, the design of the lower limb of the limb mechanism A is completed. The limb mechanism C is the same as the limb mechanism A, and its drawing is obtained by replacing the letter A with C. The limb mechanisms B and D are identical, and differ from the limb mechanisms A and C only in the angle θ. FIG. 4A-4B, i.e., the plan views of the limb mechanisms B and D, is obtained by replacing the parameter θ1 shown in FIG. 3A-3B with θ2 and then following the above design process. The labeling letter in FIG. 4A-4B is B, which represents the limb mechanism B. When the letter B is replaced with D, the drawing represents the limb mechanism D.

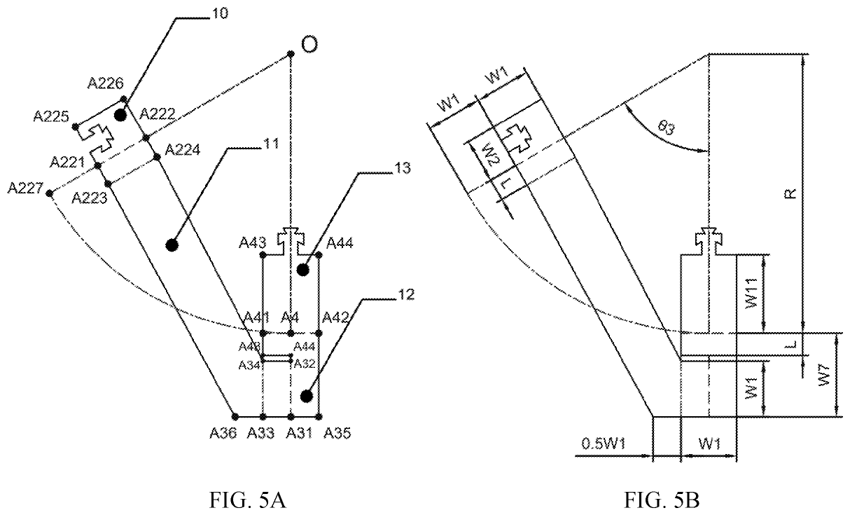

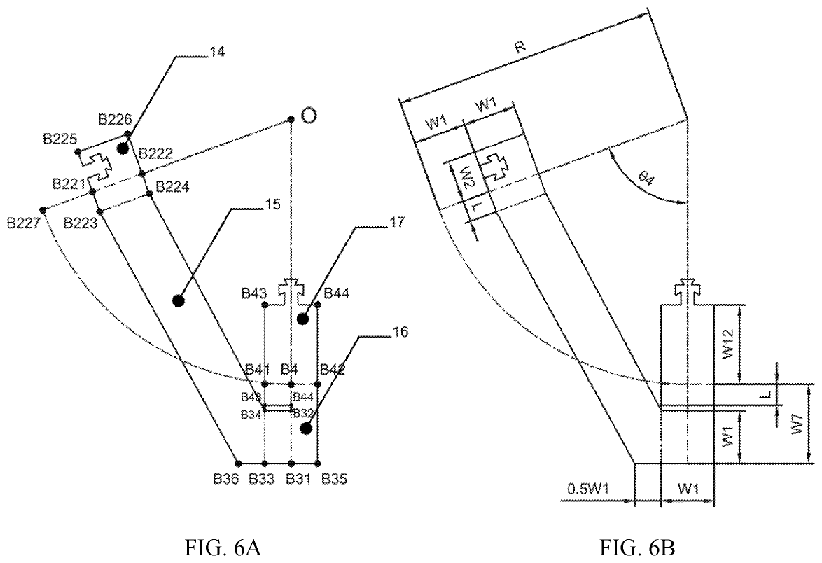

FIGS. 5A-5B are plan views of upper limbs of the limb mechanism A and the limb mechanism C, and FIGS. 6A-6B are plan views of upper limbs of the limb mechanism B and the limb mechanism D. FIGS. 5A-5B and 6A-6B have most dimensions the same, with the only difference being the different angles of θ3 and θ4 (θ3 and θ4 correspond to α3 in the theoretical model). Therefore, only FIGS. 5A-5B are used to introduce the upper limb.

In FIGS. 5A-5B, the upper limb includes tenth link 10, eleventh link 11, twelfth link 12, and thirteenth link 13. The design method of the thirteenth link 13 is as follows. Starting from the center point O, the radius R is extended downward to point A4. From the point A4, half of the link width W1 is measured to the left and right respectively, and points A41 and A42 are thus obtained. A dashed line is drawn to connect the point A41 and the point A42 to form a hinge. The points A41 and A42 are translated upward by distance W11 to obtain points A43 and A44. Solid lines are drawn to connect the point A41 to the point A43 and the point A43 to the point A44. A male connector is disposed between the point A43 and the point A44, and this connector is connected to the first link 1 during assembly.

The design method of the twelfth link 12 is as follows. The points A41 and A4 are translated downward by distance L to obtain the points A43 and A44. Solid lines are drawn to connect the point A41 to the point A43 and the point A43 to the point A44. The points A41, A4, and A42 are translated downward by distance W7 to obtain points A33, A31, and A35. Solid lines are drawn to connect the point A42 to the point A35 and the point A33 to the point A35. The points A33 and A31 are translated upward by distance W1 to obtain points A34 and A32. Solid lines are drawn to connect the point A44 to the point A32 and the point A32 to point A34. A dashed line is drawn to connect the point A31 and the point A32 to form a hinge.

The design method of the eleventh link 11 is as follows. The point A4 is rotated clockwise by angle θ3 about the point O to obtain point A227. Distance W1 is measured from point A227 along the direction of dash-dotted line A227-O, and point A221 is obtained. Distance W1 is measured from point A221 along the direction of the dash-dotted line A227-O, and point A222 is obtained. A dashed line is drawn to connect the point A221 and the point A222 to form a hinge. The dashed line A221-A222 is translated rightward by distance L along a direction perpendicular to dash-dotted line O-A222, and dash-dotted line A223-A224 is thus obtained. Half of the link width W1 is measured to the right from the point A33 to obtain point A36. Solid lines are drawn to connect point A224 to the point A34, point A223 to the point A36, and the point A36 to the point A33.

The design method of the tenth link 10 is as follows. The dashed line A221-A222 is translated leftward by distance W2 along the direction perpendicular to the dash-dotted line O-A222, and solid line A 225-A226 is obtained. A solid line is drawn to connect point A226 to point A222. A female connector is disposed between point A225 and the point A221, and this connector is connected to the seventh link 7 during assembly. After connection is completed, the point A227 coincides with the point A211, the point A221 coincides with the point A212, and the point A225 coincides with the point A216.

Thus, the design of the upper limb of the limb mechanism A is completed. The limb mechanism C is the same as the limb mechanism A, and its drawing is obtained by replacing the letter A with C. The limb mechanisms B and D are identical, and differ from the limb mechanisms A and C only in the angle θ. FIGS. 6A-6B, i.e., the plan views of the upper limbs of the limb mechanisms B and D, is obtained by replacing the parameter 03 shown in FIGS. 5A-5B with 04 and then following the above design process. The labeling letter in FIGS. 6A-6B is B, which represents the limb mechanism B. When the letter B is replaced with D, the drawing represents the limb mechanism D.

Values of the above parameters can be selected arbitrarily on the premise that processing can be realized. In this embodiment, to achieve a small robot volume, a large working space, and favorable transmission efficiency, the preferred parameters are set as follows: W1=5 mm, W11=7mm, W12=7.5 mm, W13=6 mm, W14=8 mm, W3=10 mm, W4=5 mm, W5=3 mm, W6=8 mm, W 7=7.5 mm, L=2 mm, r=0.9 mm, R=25 mm, θ1=45°, θ2=70°, θ3=60°, and θ4=70°. The theoretical model parameters corresponding to the above fabrication parameters are listed below. For the limb mechanisms A and C: α2=45°, α3=60°, L1=14 mm, and L2=2.5 mm. For the limb mechanisms B and D: α2=70°, α3=70°, L1=14 mm, and L2=2.5 mm. For all limb mechanisms, R=25 mm, and α1=15°. As described previously, the value of angle al is guaranteed by the connection between the metal base and the input link of the fixed platform.

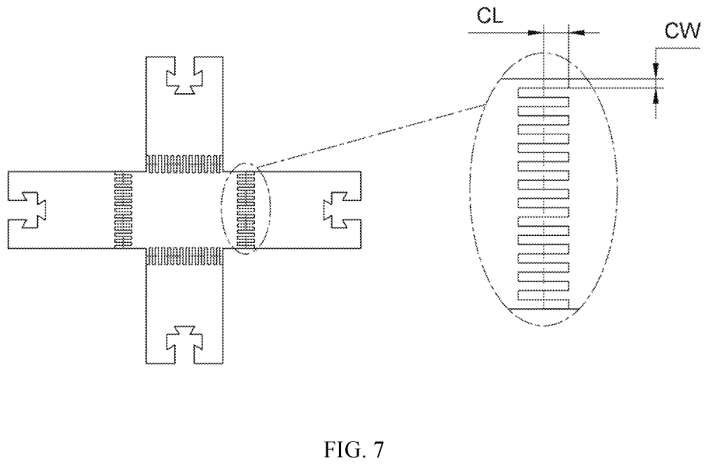

The dashed lines in FIGS. 2A to 6B are defined as flexible hinges in the above description. To improve the rigidity of the hinges, rectangular grooves are etched into the rigid material. Mutual meshing between rigid parts is enabled during rotation around the flexible hinges, and the rigidity of the hinges is enhanced accordingly. The rectangular grooves are illustrated by taking the moving platform mechanism as an example in FIG. 7, where length CL and width CW of the rectangular grooves are marked. The length CL of the rectangular groove is determined by the thickness of the rigid material and the rotation angle range of the hinge. Its value is limited to within three times the thickness of the rigid material, and is generally equal to the thickness of the rigid material. The width CW of the rectangular groove is determined by the thickness of the rigid material, the hinge length, processing precision, and other factors. Its value is greater than one-third of the thickness of the rigid material and less than one-third of the length of the hinge. In this embodiment, the preferred values are set as CL=0.55 mm and CW=0.2 mm.

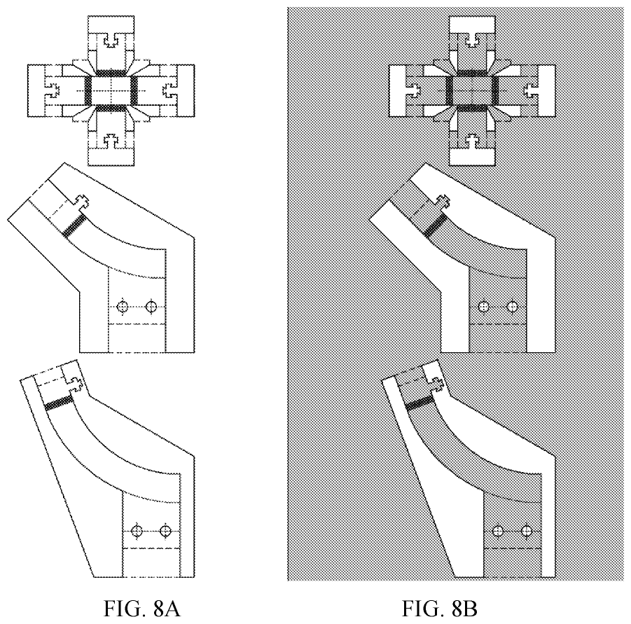

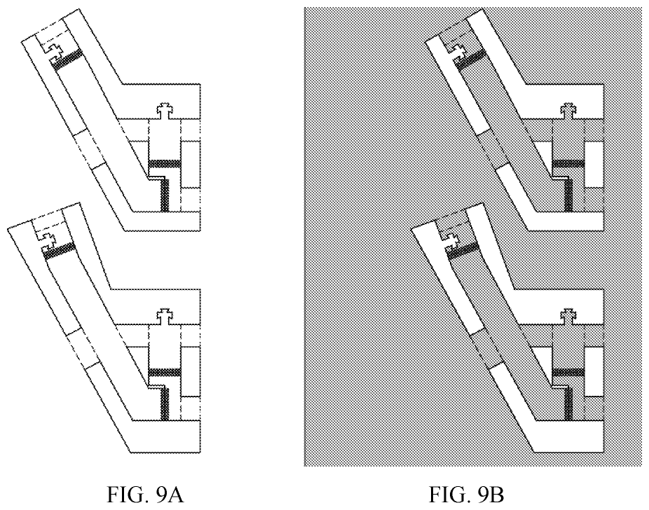

As shown in FIGS. 8A-8B and 9A-9B, in view of the processing and fabrication requirements for the smart composite material, the relative positions of all parts of the miniature robot are fixed on the sheet and shall not be separated therefrom during processing, so as to facilitate the subsequent bonding process and guarantee processing precision. Supplementary design is therefore applied to the design drawings to satisfy the above process requirements. In FIGS. 8A and 9A, connection structures between parts and the sheet, which are named “island chains”, are added on the basis of FIGS. 2A to 7. These island chains in the figures are divided by pairs of dashed lines. After the bonding process is completed, the formed smart composite components are separated from the sheet by cutting along the dashed lines. In FIGS. 8B and 9B, the removed portions of the sheet are represented by white regions, and the remaining parts after processing are represented by shaded regions. All parts are connected to the substrate sheet via the island chains.

The length of the dashed lines is defined as length DL of the island chain, and the spacing between each pair of dashed lines is defined as width DW of the island chain. The length DL of the island chain is determined by the strength and thickness of the rigid material as well as the geometric dimensions of the parts, and is generally greater than one-fifth of the length of the connected part. The width of the island chain is determined according to processing convenience, and is generally smaller than the maximum feature dimension of the connected part.

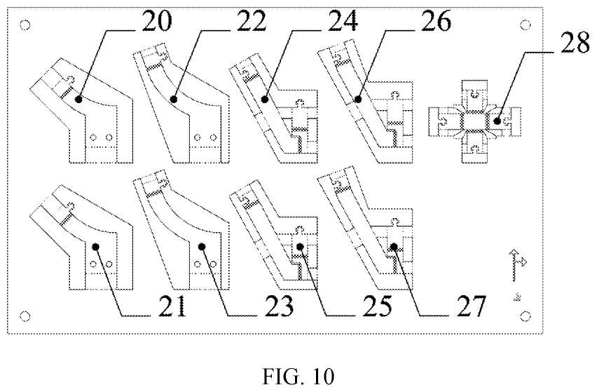

FIG. 10 presents the design drawing of nine parts required for the fabrication of a 4DoF miniature surgical robot. In this figure, part 20 is the lower limb of the limb mechanism A, and part 21 is the lower limb of the limb mechanism C, corresponding to FIGS. 3A-3B. Part 22 is the lower limb of the limb mechanism B, and part 23 is the lower limb of the limb mechanism D, corresponding to FIGS. 4A-4B. Part 24 is the upper limb of the limb mechanism A, and part 25 is the upper limb of the limb mechanism C, corresponding to FIGS. 5A-5B. Part 26 is the upper limb of the limb mechanism B, and part 27 is the upper limb of the limb mechanism D, corresponding to FIGS. 6A-6B. Part 28 is the moving platform of the robot, which corresponds to FIGS. 2A-2B.

In an embodiment of the present disclosure, the fabrication and assembly procedures of each mechanism are described as follows.

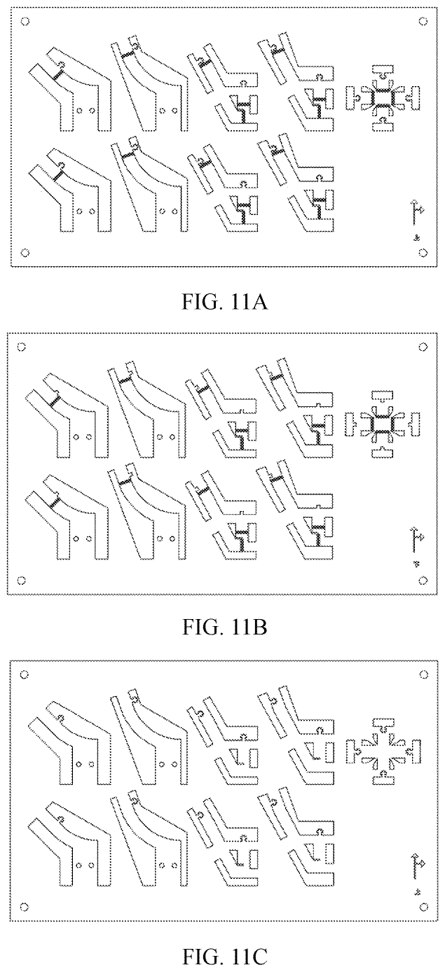

FIGS. 11A-11C are fabrication drawings of the 4DoF miniature surgical robot. On the basis of FIG. 10, the dashed lines for separating the robot from the substrate sheet are hidden. FIG. 11A is adopted for processing an upper rigid material layer and an upper adhesive material layer. FIG. 11B is adopted for processing a lower rigid material layer and a lower adhesive material layer. FIG. 11C is adopted for processing an intermediate flexible material layer. Among the above drawings, differences between FIG. 11A and FIG. 11B are reflected in the symmetry of hinge grooves about the hinge center line, the envelope configuration of male and female connectors, and the direction marks on the right side. FIG. 11C shows an intermediate layer that provides a rotation function, but presents no hinges or rectangular grooves, compared with the other two drawings.

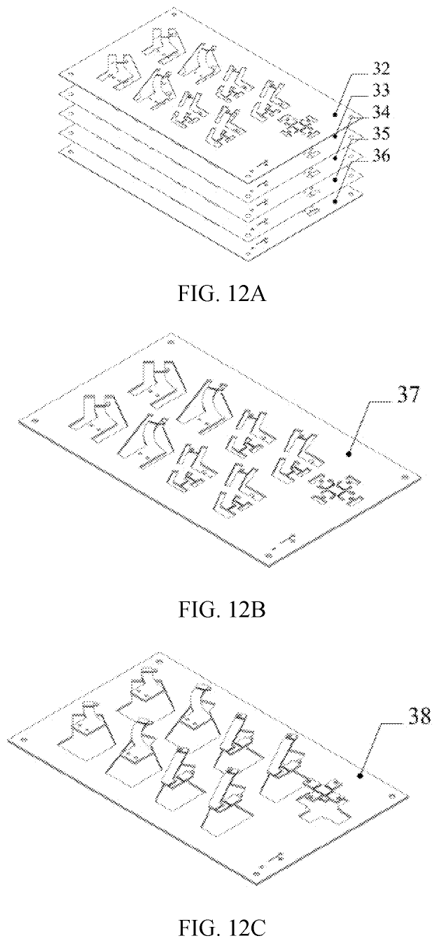

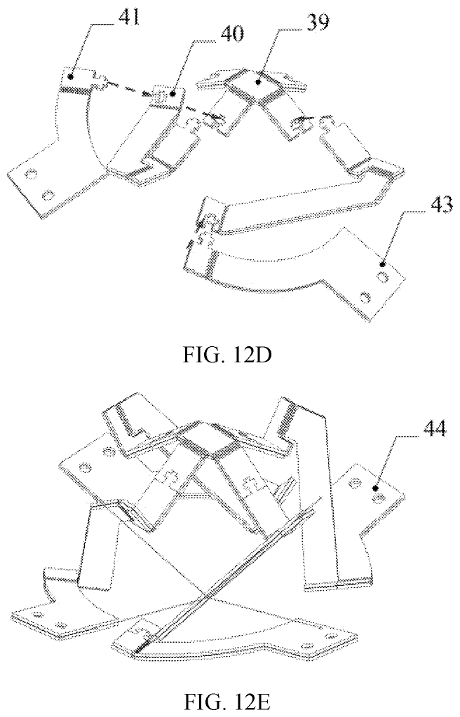

FIGS. 12A-12E show the actual fabrication and assembly flowchart of the 4DoF miniature surgical robot. FIGS. 12A-12C represent the fabrication procedures, and FIGS. 12D-12E represent the assembly procedures.

The fabrication procedures are as follows. a. Materials 32, 34, and 36 are bonded together using materials 33 and 35. b. Material 37 is formed into a five-layer smart composite structure. At this stage, all part structures of the robot are fabricated and connected to the substrate via island chains. c. All parts for assembling the 4DoF miniature surgical robot are separated from the substrate when the material 37 is cut along the dashed lines. Material 38 is the residual substrate sheet after the parts are separated. In FIG. 12A, the materials 32 and 33 correspond to FIG. 11A, the material 34 corresponds to FIG. 11C, and the sheets 35 and 36 correspond to FIG. 11B. The fabrication of all parts of the 4DoF miniature surgical robot is thus completed.

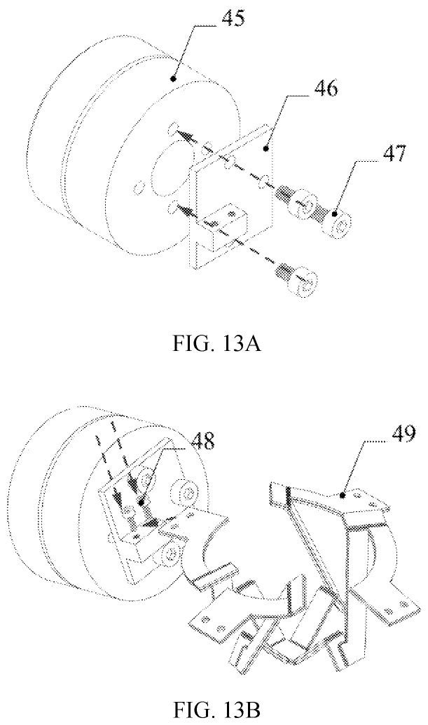

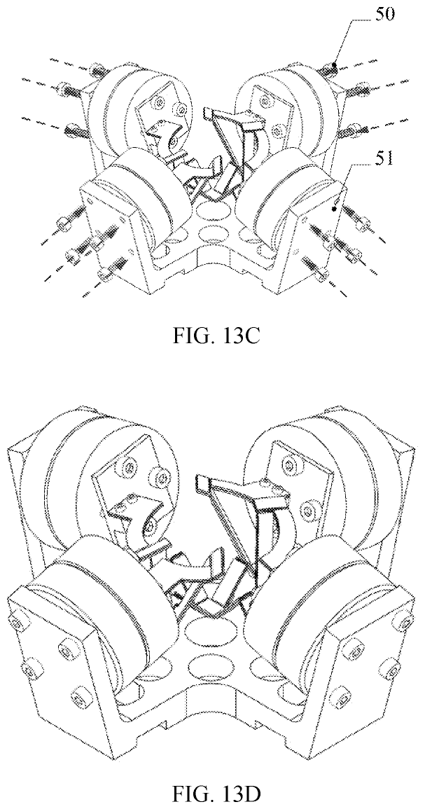

The assembly procedures are as follows. d. Part 39 corresponds to FIGS. 2A-2B and is configured as the moving platform mechanism of the 4DoF miniature surgical robot. Part 40 corresponds to FIGS. 5A-5B and is configured as the upper limb of the limb mechanism A. Part 41 corresponds to FIGS. 3A-3B and is configured as the lower limb of the limb mechanism A. Part 42 corresponds to FIGS. 6A-6B and is configured as the upper limb of the limb mechanism D. Part 43 corresponds to FIGS. 4A-4B, with the labeling letter B replaced by D, and is configured as the lower limb of the limb mechanism D. As shown in FIG. 12D, the male connectors of the part 40 and the part 42 are inserted into the female connectors of the part 39. The male connector of the part 41 is inserted into the female connector of the part 40. The male connector of the part 43 is inserted into the female connector of the part 42. e. After the insertion connection of the limb mechanisms A, B, C, and D is completed in accordance with the method shown in FIG. 12D, the connection positions are secured with adhesive and fully cured, and the robot body is thus obtained, thereby completing the assembly of the 4DoF miniature surgical robot. The spatial position of the fixed platform of the robot is not fixed yet. It is subsequently positioned by a metal base (FIGS. 13A-13D), and rotary driving power is thereby delivered to the four limb mechanisms.

Power for controlling the miniature robot is transmitted via the lower limbs of the four limb mechanisms (the sixth link 6 of the limb mechanisms A and C, and the eighth link 8 of the limb mechanisms B and D). When the pose of the rotation axis of each input link is fixed in compliance with the parameter requirements of the theoretical model, angular control of the four input links by their respective rotary drive mechanisms enables 4DoF control of the moving platform (the fifth link 5) within the working space of the miniature surgical robot.

Therefore, on the basis of the 4DoF miniature surgical robot with an RCM in the above embodiments, a control system for the 4DoF miniature surgical robot with an RCM is further provided. The control system includes the 4DoF miniature surgical robot with an RCM described in the above embodiments and four independent rotary drive mechanisms. The lower limb of each limb mechanism is provided with one rotary drive mechanism fixed to the base, so as to drive the corresponding limb mechanism to rotate about the associated rotation center axis.

The above angle control function can be realized by various conventional rotary drive mechanisms according to operating conditions and precision requirements, such as piezoelectric ceramic drive, motor drive, wire drive, electromagnetic drive, friction drive, shape memory alloy drive, and flexible material deformation drive. Mechanical transmission and motor actuation are adopted for demonstration in this embodiment. Each rotary drive mechanism includes a drive motor and a transmission plate. The transmission plate is mounted on a drive shaft of the drive motor, and its plate surface is perpendicular to the drive shaft. A mounting plane for the protruding portion of the sixth link is provided on the transmission plate via a boss structure. The specific assembly configuration of the rotary drive mechanism is presented as follows.

FIGS. 13A-13D illustrate the miniature surgical robot and its drive and transmission system. FIG. 13A is an assembly view of the motor and the transmission plate. In this figure, part 45 is a micro servo motor, part 46 is the transmission plate, and parts 47 are No. 1 bolts. The transmission plate is provided with three through holes for cooperating with the No. 1 bolts to fix the motor and two bolt holes for connecting and fixing the miniature surgical robot. The transmission plate is configured to connect the micro servo motor and the miniature surgical robot. The rotation axis of the input link of the robot coincides with the rotation axis of the motor, such that the structural requirements of the robot specified by the theoretical model are satisfied. FIG. 13B is an assembly view of the miniature surgical robot and the transmission plate. In this figure, parts 48 are No. 2 bolts, and part 49 is the inverted body of the miniature surgical robot. As illustrated, the robot is connected to the transmission plate by the No. 2 bolts which penetrate two through holes formed in the input link. FIG. 13C is an assembly view of the motor and the base. Parts 50 are No. 3 bolts, and part 51 is the base. Four inclined surfaces are formed on the base to secure the motors in compliance with the requirements of the theoretical model. Four through holes are provided on each inclined surface, and the motors are fastened to the base via the No. 3 bolts inserted into these through holes, thereby obtaining FIG. 13D. FIG. 13D is a schematic view presenting the state after completion of the above assembly procedures. The 4DoF miniature surgical robot system is thus fully assembled. The fixed platform of the robot is secured by the metal base via the motors and transmission plates. At this stage, the actual structural parameters of the robot are consistent with the parameters of the theoretical model. Precise control of the moving platform of the robot is realized through angle control of the miniature servo motors, and the actuator mounted on the moving platform is thereby enabled to complete target tasks.

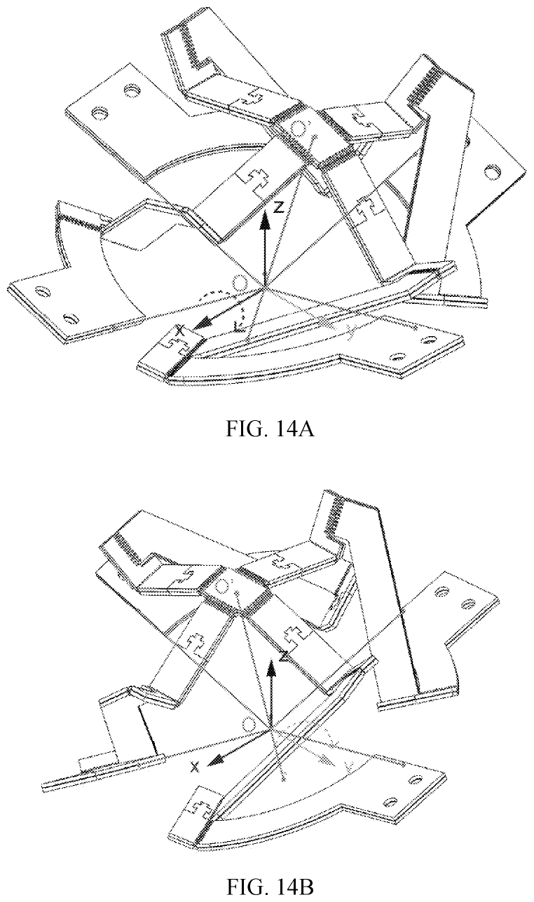

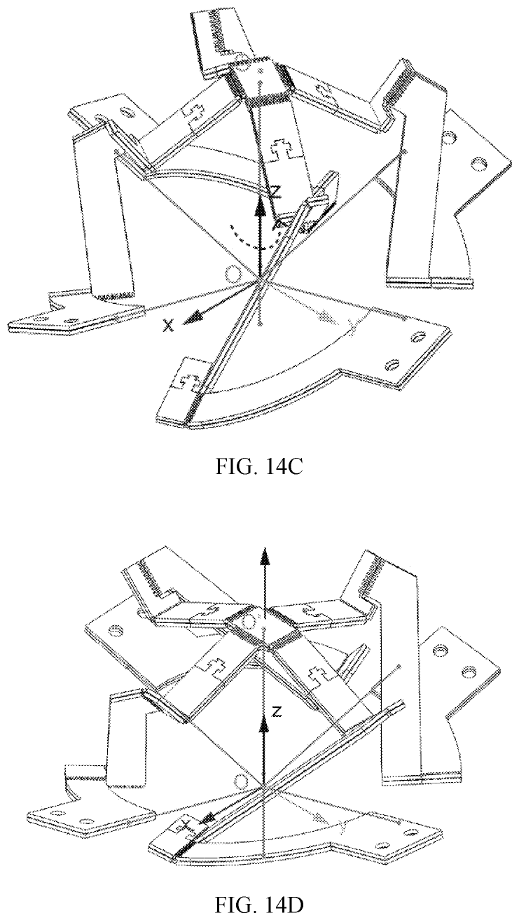

FIGS. 14A-14D are schematic diagrams of motion degrees of freedom of the moving platform of the miniature robot. In this figure, origin O of a global coordinate system coincides with the RCM point of the robot. An x-axis is parallel to the distribution direction of the limb mechanism A, and a y-axis is parallel to the distribution direction of the limb mechanism D. Point O′ is a geometric center of the moving platform of the robot. FIGS. 14A-14D demonstrate respectively the rotational degree of freedom of the moving platform of the miniature surgical robot about the x-axis, the rotational degree of freedom about the y-axis, the rotational degree of freedom about a z-axis, and the translational degree of freedom along the z-axis. It can be seen that a straight line passing through the point O′ and perpendicular to the moving platform always intersects the point O, so the point O is configured as an RCM point of the miniature robot.

In addition, in some embodiments of the present disclosure, the area on the moving platform (the fifth link 5) is designed with specific profiles, such that different devices can be mounted on the miniature surgical robot to adapt to various operating conditions. Examples of such devices include blades, miniature cameras, injection needles, lenses, and suction cups. The robot can thereby accomplish multiple tasks including cutting, positioning imaging, puncture and injection, laser control, as well as suction and gripping. As shown in FIGS. 15A-15C, three embodiments provided with a blade, a miniature camera, and an injection needle are presented in the present disclosure. In FIG. 15A, a model of the miniature surgical robot fitted with a blade for cutting operations is shown. In FIG. 15B, a model of the miniature surgical robot fitted with a miniature camera for imaging the surgical environment is shown. In FIG. 15C, a model of the miniature surgical robot fitted with an injection needle for puncture operations is shown.

The theoretical model, specific configuration, and processing method of the 4DoF miniature surgical robot with four limb mechanisms are illustrated in FIGS. 1 to 14D. Nevertheless, this is merely a preferred implementation, and the present disclosure is not limited thereto. The number of the limb mechanisms involved in the present disclosure can be adjusted reasonably according to actual demands, and two or more limb mechanisms are acceptable. Accordingly, the configuration of the moving platform mechanism is adjusted adaptively in accordance with the number of the limb mechanisms. The moving platform mechanism shall meet the following basic requirements. It includes a central link having a polygonal planar shape and limb links which are connected to sides of the central link via hinges. The central link functions as the moving platform for mounting surgical instruments. Both the number of the limb links and the number of sides of the polygon are not less than the number of the limb mechanisms. Furthermore, a mismatch may exist between the number of the limb mechanisms and the number of the rotary drive mechanisms. For the control system of the 4DoF miniature surgical robot in the present disclosure, the mounting sequence of the rotary drive mechanisms on each of the limb mechanisms is specified as follows. Firstly, one rotary drive mechanism fixed to the base is mounted on the lower limb of each limb mechanism, so as to drive the corresponding limb mechanism to rotate about the associated rotation center axis. The number of the rotary drive mechanisms is generally equal to the number of the limb mechanisms. A larger number is also allowed, yet the extra rotary drive mechanisms will become redundant. The minimum number of the limb mechanisms provided on the robot is two. While four limb mechanisms are adopted in the above embodiments, configurations with two, three, five, or more limb mechanisms are also applicable. In other words, the number of the rotary drive mechanisms is not necessarily matched with that of the limb mechanisms. Four limb mechanisms and four rotary drive mechanisms are provided respectively, and a one-to-one driving correspondence is established between the rotary drive mechanisms and the limb mechanisms as described in the above embodiments. One rotary drive mechanism fixed to the base is mounted on the lower limb of each limb mechanism, and the axis of the output shaft of the rotary drive mechanism coincides with the rotation center axis provided on the lower limb. If the number of rotary drive mechanisms is greater than the number of limb mechanisms, apart from the rotary drive mechanism mounted on the lower limb of each limb mechanism and fixed to the base, the remaining rotary drive mechanisms are respectively mounted on the upper limbs of mutually different limb mechanisms, each remaining rotary drive mechanism being configured to drive the limb mechanism on which it is mounted to rotate about the corresponding seventh hinge. The rotary drive mechanism mounted on the upper limb of the limb mechanism can move spatially together with the rotary drive mechanism, avoiding the need for mounting via the base.

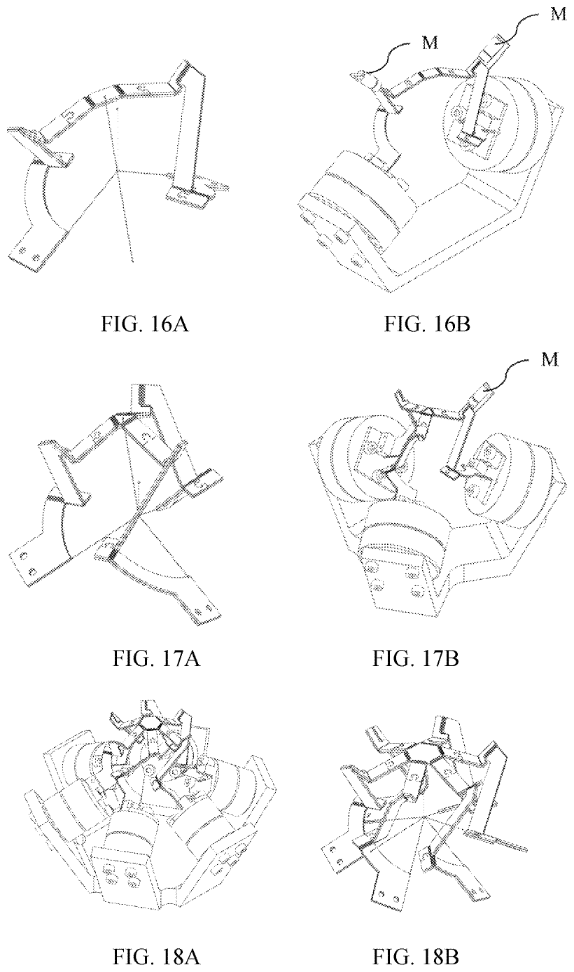

As shown in FIGS. 16A-16B, the main body of the 4DoF miniature surgical robot provided with only two limb mechanisms is presented in the left drawing, and the control system of the 4DoF miniature surgical robot assembled on the base is presented in the right drawing. Four rotary drive motors are provided in the control system of the 4DoF miniature surgical robot. Two of the rotary drive motors are fixed to the base to drive the two limb mechanisms to rotate about their respective rotation center axes. The other two rotary drive motors marked M in the drawing are mounted on the upper limbs of the two limb mechanisms to drive the corresponding limb mechanisms to rotate about the seventh hinges thereof.

As shown in FIGS. 17A-17B, the main body of the 4DoF miniature surgical robot provided with only three limb mechanisms is presented in the left drawing, and a control system of the 4DoF miniature surgical robot assembled on the base is presented in the right drawing. Four rotary drive motors are provided in the control system of the 4DoF miniature surgical robot. Three of the rotary drive motors are fixed to the base to drive the three limb mechanisms to rotate about their respective rotation center axes. The remaining one rotary drive motor marked M in the drawing is mounted on the upper limb of any one limb mechanism to drive the corresponding limb mechanism to rotate about the seventh hinge thereof.

As shown in FIGS. 18A-18B, the main body of the 4DoF miniature surgical robot provided with six limb mechanisms is presented in the left drawing, and a control system of the 4DoF miniature surgical robot assembled on the base is presented in the right drawing. Six rotary drive motors are fixed to the base in the control system of the 4DoF miniature surgical robot to drive the six limb mechanisms to rotate about their respective rotation center axes. In this configuration, additional rotary drive motors can be further mounted. Such extra motors can be mounted on the upper limbs of any limb mechanisms to drive the corresponding limb mechanisms to rotate about the seventh hinges thereof, yet these motors are redundant and can be omitted.

In addition, the specific configurations of each limb mechanism illustrated in FIGS. 8A to 11C are merely preferred embodiments of the present disclosure. The structures of the upper limb and the lower limb can be adjusted or simplified on the basis of the above preferred embodiments.

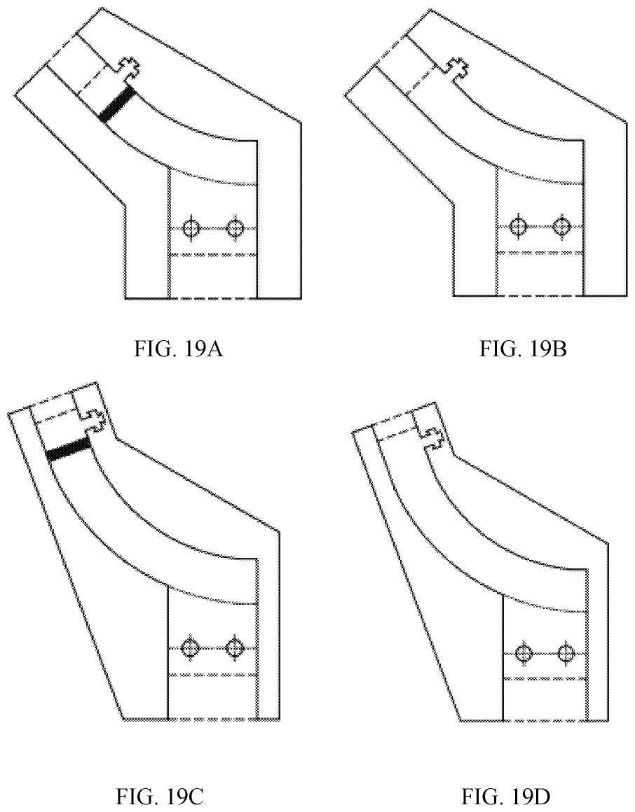

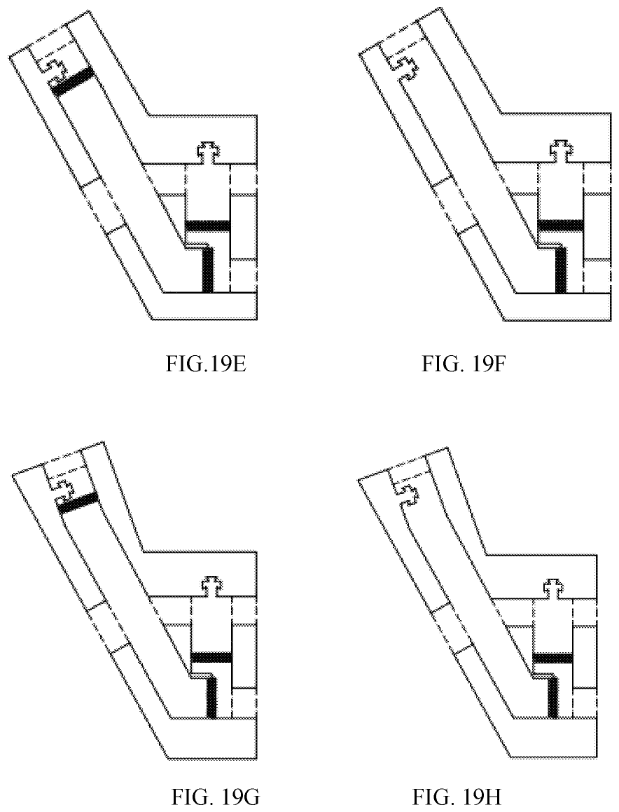

In a variant embodiment of the present disclosure, the fifth hinge and the sixth hinge of each limb mechanism are not required to be provided simultaneously, and either one can be selectively omitted. As shown in FIGS. 19A-19H, FIGS. 19A,19C, 19E, and 19G illustrate the condition where both the fifth hinge and the sixth hinge are provided on four links, while FIGS. 19B, 19D, 19F, and 19H illustrate the condition where either the fifth hinge or the sixth hinge is omitted on the four links. If a hinge is omitted, the two links on two sides of the hinge are connected fixedly. If a hinge is retained, the two links on two sides of the hinge are connected rotatably via the hinge. It shall be specially noted that the fixed connection between two links can be realized by assembling male and female connectors, or by integral forming during processing. No physical joint is formed at the connection position in the integral forming mode, and the two links are fabricated as an integral component.

In another variant embodiment, one side of the protruding portion of the sixth link is not necessarily collinear with one side of the second straight section. When they are not collinear, a straight line defined by the side of the second straight section away from the annular section is configured as the rotation center axis of the lower limb during driving. In summary, in the present disclosure, each lower limb shall comply with the following basic requirements. The lower limb is integrally formed by sixth link 6 and seventh link 7. The sixth link 6 includes a main body portion and a protruding portion. The main body portion is continuously formed by a first straight section, an annular section, and a second straight section, all having the same width. The seventh link 7 is connected to an outer end portion of the first straight section fixedly or rotatably via a fifth hinge. The protruding portion is provided on an outer annular side of the main body portion and provided with mounting holes connectable to an external drive mechanism. A straight line defined by a side of the second straight section away from the annular section is configured as the rotation center axis of the lower limb when the lower limb is driven. Each upper limb shall comply with the following basic requirements. The upper limb is integrally formed by tenth link 10, eleventh link 11, twelfth link 12, and thirteenth link 13. One end of the tenth link 10 is connected to one end of the eleventh link 11 fixedly or rotatably via the sixth hinge. The other end of the eleventh link 11 is rotatably connected to one end of the twelfth link 12 via the seventh hinge. The other end of the twelfth link 12 is rotatably connected to the thirteenth link 13 via the eighth hinge. The thirteenth link 13 is fixedly assembled with the corresponding limb link on the moving platform mechanism via male and female connectors. The lower limb and the upper limb are fixedly assembled with each other via a set of male and female connectors respectively provided on the seventh link 7 and the tenth link 10.

In addition, it should be noted that, the parameters of all components of the 4DoF miniature surgical robot shall be reasonably constrained and optimized according to actual application demands. In accordance with the theoretical model and geometric relationships, each limb mechanism of the 4DoF miniature surgical robot is defined by six structural parameters, namely R, α1, α2, α3, L1, and L2. In the present disclosure, R is defined as equivalent radius; α1, α2, and α3 are defined as a first attitude angle, a second attitude angle, and a third attitude angle respectively; and L1 and L2 are defined as a first hinge spacing and a second hinge spacing, respectively. The detailed definitions of the six structural parameters for each limb mechanism are specified below for better understanding.

Since the fifth hinge and the sixth hinge are not necessarily provided simultaneously, the retained fifth hinge or sixth hinge is defined as a designated hinge for descriptive purposes (when both the fifth hinge and the sixth hinge are retained, they coincide with each other). An intersection point of the designated hinge and the rotation center axis is defined as a first center point. An intersection point of the designated hinge and the seventh hinge is defined as a second center point. The equivalent radius R of the lower limb is defined as either a distance from the first center point to a farthest end of the designated hinge, or a distance from the first center point to a farthest end of the side of the second straight section away from the annular section. The equivalent radius R of the upper limb is defined as a distance from the second center point to the eighth hinge. An angle between the rotation center axis and the horizontal plane is defined as the first attitude angle al. An angle between the designated hinge and the rotation center axis is defined as the second attitude angle α2. An angle between the designated hinge and the seventh hinge is defined as the third attitude angle α3. The first hinge spacing L1 is defined as a distance from the eighth hinge to the hinge on the moving platform that is connected to the current limb mechanism. The second hinge spacing L2 is defined as half a distance between the hinge on the moving platform that is connected to the current limb mechanism and a symmetric hinge thereof.

In the present disclosure, all the limb mechanisms assembled on the central link of the 4DoF miniature surgical robot shall satisfy the following five basic constraints.

In a first basic constraint, each limb mechanism includes at least one of the fifth hinge and the sixth hinge; and, for any limb mechanism that includes both hinges, the axes of the fifth hinge and the sixth hinge coincide with each other.

In a second basic constraint, the axis of the seventh hinge and the axis of the eighth hinge of each limb mechanism are perpendicular to each other.

In a third basic constraint, the second attitude angle α2 and the third attitude angle α3 of each limb mechanism are less than 180°.

In a fourth basic constraint, the eighth hinge of each limb mechanism is parallel to the hinge on the moving platform that is connected to the current limb mechanism.

In a fifth basic constraint, all first center points and all second center points of all the limb mechanisms coincide at one and the same point.

The above five basic constraints are mandatory for miniature surgical robots with any number of limb mechanisms. For different practical miniature surgical robots, additional constraints can be formulated according to practical application requirements. In the embodiments of the present disclosure, for the miniature surgical robot under the theoretical configuration shown in FIG. 1, a total of 4×6=24 structural parameters are involved for a total of four limb mechanisms. To achieve a large working space and excellent transmission performance, these structural parameters shall further satisfy the following four optimization constraints.

In a first optimization constraint, the equivalent radius R of the lower limbs and upper limbs of the four limb mechanisms is set to the same value.

In a second optimization constraint, the parameters of each pair of opposite limb mechanisms are identical. Specifically, the second attitude angle α2, the third attitude angle α3, the first hinge spacing L1, and the second hinge spacing L2 of the first limb mechanism are equal to those of the third limb mechanism, and the second attitude angle α2, the third attitude angle α3, the first hinge spacing L1, and the second hinge spacing L2 of the second limb mechanism are equal to those of the fourth limb mechanism.

In a third optimization constraint, by taking the equivalent radius R as a characteristic dimension of the mechanisms, the first hinge spacing L1 and the second hinge spacing L2 of each of the four limb mechanisms are controlled within the range of 0.2 to 2 times the equivalent radius R.

In a fourth optimization constraint, the second attitude angle α2 and the third attitude angle α3 of each of the four limb mechanisms are less than 90°.

It shall be specially explained that the first attitude angle al of each limb mechanism is formed only after the robot is assembled with the drive mechanisms, so relevant constraints cannot be applied before assembly. After the assembly of the robot with the drive mechanism is completed, the first attitude angle al shall also comply with the second optimization constraint. That is, the first attitude angle al of the first limb mechanism is equal to that of the third limb mechanism, and the first attitude angle al of the second limb mechanism is equal to that of the fourth limb mechanism. For the robot with four limb mechanism in the above embodiments, it is preferred that the first attitude angles al of the four limb mechanisms are set to the same value.

The above described are merely preferred embodiments of the present disclosure, and are not intended to limit the present disclosure. Various changes and transformations can be made by persons of ordinary skill in the relevant technical field without departing from the spirit and scope of the present disclosure. All technical solutions obtained by means of equivalent substitution or equivalent transformation should fall within the protection scope of the present disclosure. The listing of claims will replace all prior versions and listings of claims in the application.

What is claimed is:

1. A four-degrees-of-freedom (4DoF) miniature surgical robot with a remote center-of-motion (RCM), comprising: a moving platform mechanism fabricated from a layered composite material by planar processing and at least two limb mechanisms, wherein
the moving platform mechanism comprises a central link having a polygonal planar shape and limb links connected to sides of the central link via hinges; the central link is configured as a moving platform for mounting a surgical instrument; a number of the limb links and a number of sides of the polygon are greater than or equal to a number of the limb mechanisms;
the limb mechanisms share a same configuration and are each formed by mating assembly of an upper limb and a lower limb;
the lower limb is integrally formed by a sixth link and a seventh link; the sixth link comprises a main body portion and a protruding portion; the main body portion is continuously formed by a first straight section, an annular section, and a second straight section, all having a same width; the seventh link is connected to an outer end portion of the first straight section fixedly, or rotatably via a fifth hinge; the protruding portion is provided on an outer annular side of the main body portion and is provided with a mounting hole connectable to an external drive mechanism; and a straight line defined by a side of the second straight section away from the annular section is configured as a rotation center axis of the lower limb when the lower limb is driven;
the upper limb is integrally formed by a tenth link, an eleventh link, a twelfth link, and a thirteenth link; the tenth link is connected to a first end of the eleventh link fixedly, or rotatably via a sixth hinge; a second end of the eleventh link is rotatably connected to a first_end of the twelfth link via a seventh hinge; a second end of the twelfth link is rotatably connected to the thirteenth link via an eighth hinge; and the thirteenth link is fixedly assembled with a corresponding limb link on the moving platform mechanism via male and female connectors;
the lower limb and the upper limb are fixedly assembled via a set of male and female connectors respectively provided on the seventh link and the tenth link;
the limb mechanisms assembled on the central link satisfy following five constraints:
a first constraint: each limb mechanism comprises at least one of the fifth hinge and the sixth hinge; and, for any limb mechanism that comprises both hinges, axes of the fifth hinge and the sixth hinge coincide with each other;
a second constraint: axes of the seventh hinge and the eighth hinge of each of the limb mechanisms are perpendicular to each other;
a third constraint: a second attitude angle and a third attitude angle of each of the limb mechanisms are less than 180°;
a fourth constraint: the eighth hinge in each of the limb mechanisms is parallel to a hinge on the moving platform that is connected to a current limb mechanism; and
a fifth constraint: all first center points and all second center points of all the limb mechanisms coincide at one and the same point; and
in each of the limb mechanisms, the provided fifth hinge or sixth hinge is configured as a designated hinge, an intersection point of the designated hinge and the rotation center axis is configured as the first center point, an intersection point of the designated hinge and the seventh hinge is configured as the second center point, an angle between the designated hinge and the rotation center axis is configured as the second attitude angle, and an angle between the designated hinge and the seventh hinge is configured as the third attitude angle.

2. The 4DoF miniature surgical robot according to claim 1, wherein in the moving platform mechanism, the central link is a fifth link shaped as a rectangle; the fifth link is connected to four limb links: a first link, a second link, a third link, and a fourth link; a first end of the first link, a first end of the second link, a first end of the third link, and a first end of the fourth link are respectively rotatably connected to the fifth link via a first hinge, a second hinge, a third hinge, and a fourth hinge, and a second end of the first link, a second end of the second link, a second end of the third link, and a second end of the fourth link are respectively fixedly connected to a first limb mechanism, a second limb mechanism, a third limb mechanism, and a fourth limb mechanism in a manner of assembling male and female connectors; and the first hinge and the third hinge are parallel to each other, the second hinge and the fourth hinge are parallel to each other, and the first hinge and the second hinge are perpendicular to each other; and
in the moving platform mechanism and the four limb mechanisms connected to the moving platform mechanism, a combined mechanism of the first link and the first limb mechanism and a combined mechanism of the third link and the third limb mechanism are symmetrically disposed on two sides of the moving platform, and a combined mechanism of the second link and the second limb mechanism and a combined mechanism of the fourth link and the fourth limb mechanism are symmetrically disposed on two sides of the moving platform.

3. The 4DoF miniature surgical robot-with according to claim 2, wherein a robot body assembled on four rotary drive mechanisms satisfies following four constraints:
a first constraint: the lower limb and the upper limb of each of the four limb mechanisms have a same equivalent radius;
a second constraint: the first limb mechanism and the third limb mechanism have a same second attitude angle, third attitude angle, first hinge spacing, and second hinge spacing, and the second limb mechanism and the fourth limb mechanism have a same second attitude angle, third attitude angle, first hinge spacing, and second hinge spacing;
a third constraint: the first hinge spacing and the second hinge spacing of each of the four limb mechanisms are 0.2 to 2 times the equivalent radius; and
a fourth constraint: the second attitude angle and the third attitude angle of each of the four limb mechanisms are less than 90°; and
in each of the limb mechanisms, a distance from the first center point to a farthest end of the designated hinge, or a distance from the first center point to a farthest end of the side of the second straight section away from the annular section, is configured as an equivalent radius of the lower limb; a distance from the second center point to the eighth hinge is configured as an equivalent radius of the upper limb; an angle between the rotation center axis and a horizontal plane is configured as the first attitude angle; a distance from the eighth hinge to the hinge on the moving platform that is connected to the current limb mechanism is configured as the first hinge spacing; and half of a distance from the hinge on the moving platform that is connected to the current limb mechanism to a symmetric hinge is configured as the second hinge spacing.

4. The 4DoF miniature surgical robot according to claim 1, wherein all links of the 4DoF miniature surgical robot are made of a layered composite material; the layered composite material comprises a flexible planar material layer configured as an intermediate layer and rigid planar material layers located on two sides of the intermediate layer; the flexible planar material layer is bonded and fixed to the rigid planar material layers by an adhesive material layer; at a hinge position between every two adjacent links, the flexible planar material layer is continuous while the rigid planar material layers are discontinuous; and
edges of the rigid planar material layers on two sides of a discontinuity position are provided with rectangular grooves at intervals, such that the rigid planar material layers interlock with each other via sawtooth edges formed by the rectangular grooves and are allowed to freely rotate about a hinge under a connecting action of the flexible planar material layer.

5. The 4DoF miniature surgical robot according to claim 4, wherein the flexible planar material layer is a flexible polymer film, a flexible gel layer, a flexible textile, or a flexible metal foil; and each of the rigid planar material layers is a rigid metal sheet, a rigid plastic sheet, a rigid glass sheet, a rigid resin sheet, a rigid wood sheet, or a rigid composite material sheet.

6. The 4DoF miniature surgical robot according to claim 1, wherein in each of the limb mechanisms, male and female connectors for connecting two links are respectively provided on the two links; a male connector of a first link of the two links is assembled into a female connector of a second link of the two links; and
the two links are bonded together by adhesive.

7. A control system for a 4DoF miniature surgical robot with an RCM, comprising the 4DoF miniature surgical robot according to claim 1 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle;
and the first attitude angle of each of the limb mechanisms is less than 90°.

8-10. (canceled)

11. A control system for a 4DoF miniature surgical robot with an RCM, comprising the 4DoF miniature surgical robot according to claim 2 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle; and the first attitude angle of each of the limb mechanisms is less than 90°.

12. A control system for a 4DoF miniature surgical robot with an RCM, comprising the 4DoF miniature surgical robot according to claim 3 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle;
and the first attitude angle of each of the limb mechanisms is less than 90°.

13. A control system for a 4DoF miniature surgical robot with an RCM, comprising the 4DoF miniature surgical robot according to claim 4 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle;
and the first attitude angle of each of the limb mechanisms is less than 90°.

14. A control system for a 4DoF miniature surgical robot with an RCM, comprising the 4DoF miniature surgical robot according to claim 5 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle;
and the first attitude angle of each of the limb mechanisms is less than 90°.

15. A control system for a 4DoF miniature surgical robot with an RCM, comprising the 4DoF miniature surgical robot according to claim 6 and at least four independent rotary drive mechanisms, wherein
the lower limb of each of the limb mechanisms is provided with one rotary drive mechanism that is fixed to a base for driving the limb mechanism to rotate about the corresponding rotation center axis; and if a number of rotary drive mechanisms is greater than a number of limb mechanisms, remaining rotary drive mechanisms are respectively mounted on the upper limbs of mutually different limb mechanisms, each remaining rotary drive mechanism being configured to drive the limb mechanism on which it is mounted to rotate about the corresponding seventh hinge; and
after each of the limb mechanisms is assembled with the rotary drive mechanism, an angle between the rotation center axis and the horizontal plane is configured as the first attitude angle; and the first attitude angle of each of the limb mechanisms is less than 90°.

16. The control system according to claim 7, wherein there are four limb mechanisms and four rotary drive mechanisms, and the rotary drive mechanisms correspond one-to-one with the limb mechanisms to form a driving relationship.

17. The control system according to claim 16, wherein the four limb mechanisms have the same first attitude angle.

18. The control system according to claim 11, wherein there are four limb mechanisms and four rotary drive mechanisms, and the rotary drive mechanisms correspond one-to-one with the limb mechanisms to form a driving relationship.

19. The control system according to claim 7, wherein each of the rotary drive mechanisms comprises a drive motor and a transmission plate; the transmission plate is assembled on a drive shaft of the drive motor and comprises a plate surface perpendicular to the drive shaft; the transmission plate provides a mounting plane for the protruding portion of the sixth link via a boss structure.

20. The control system according to claim 19 wherein the drive motor is a micro servo motor.

21. The control system according to claim 11, wherein each of the rotary drive mechanisms comprises a drive motor and a transmission plate; the transmission plate is assembled on a drive shaft of the drive motor and comprises a plate surface perpendicular to the drive shaft; the transmission plate provides a mounting plane for the protruding portion of the sixth link via a boss structure.

22. The control system according to claim 11, wherein a center of the fifth link configured as the moving platform is provided with a mounting hole, and a surgical instrument is mounted via the mounting hole.

23. The control system according to claim 22, wherein the surgical instrument comprises one or more of a blade, a miniature camera, an injection needle, a lens, and a suction cup.
